(19) United States
(12) Patent Application Publication
(10) Pub. No.: US 2002/0180605 A1
(43) Pub. Date: Dec. 5, 2002

(54) Wearable biomonitor with flexible thinned integrated circuit

(76) Inventors: Volkan H. Ozguz, Aliso Vlejo, CA (US); Abbas Khashayar, Anaheim, CA (US)

Correspondence Address: MYERS DAWES & ANDRAS LLP, Suite 1150, 19900 MacArthur Boulevard, Irvine, CA, 92612, US

(21) Appl. No.: 10/197,006

(22) Filed: Jul. 16, 2002

Related U.S. Application Data
(63) Continuation-in-part of application No. 09/190,378, filed on Nov. 10, 1998.
(60) Provisional application No. 60/065,088, filed on Nov. 11, 1997.
(60) Provisional application No. 60/305,353, filed on Jul. 16, 2001.

(51) Int. Cl. A61B 18/04

(52) U.S. Cl. 340/573.1; 174/254; 361/749; 257/700; 257/701; 257/702; 606/32

(57) ABSTRACT

A sensor system (30) has a sensor module (10) and a receiver module (45). The sensor module (10) functions as a wireless data collection device and has a flexible thin sheet of silicon (60, 65, 70) comprising circuitry (71, 72, 73), a flexible power source (105), and a flexible support substrate (55). The silicon, power source, and flexible support substrate are integrated as layers of the sensor module (10). The layers are placed together in the form of an adhesive bandage (10). A plurality of electrodes (80) are connected to the sensor module (10) and protrude from the flexible substrate (55) for contacting the skin of a subject body (20). The receiver module (45) includes one of an RF receiver with a wireless port for continuously receiving data (40), or a physical I/O port (87) to which the sensor module (10) can be physically connected for downloading stored data from the sensor module (10).

### CROSS REFERENCE TO APPLICATIONS

This application is a continuation-in-part of U.S. patent application Ser. No. 09/190,378 filed Nov. 10, 1998, entitled “Method for Thinning Semiconductor Wafers with Circuits and Wafers Made by the Same”, which claims the benefit of provisional application Ser. No. 60/065,088, filed Nov. 11, 1997, entitled “Method for Thinning Semiconductor Wafers with Circuits” and this application claims the benefit of U.S. patent application Ser. No. 60/305,353 filed Jul. 16, 2001, entitled “Biomonitor Device”, all three of which are incorporated herein by reference.

### BACKGROUND OF THE INVENTION

1. Field of the Invention

The instant invention relates generally to sensor systems for detecting physiological characteristics, and more specifically to a sensor system comprising a thin flexible ambulatory/self contained bio-sensor module in a form similar to an adhesive bandage for sensing physiologically modulated signals from the body, and a method of making such a sensor system and module.

2. Description of Prior Art and Related Information

Sensor systems have been used to detect a variety of physiological characteristics. Most of the sensor systems for detecting and recording electromagnetic signals from the body comprise sensor modules with rigid circuitry to which electrodes are connected. Use of rigid circuitry is due to the rigid nature of conventional microcircuit materials such as silicon and printed circuit board materials. The result is that the circuitry is inflexible, if not bulky, and is unsuitable for comfortably adhering and conforming to a localized portion of a human body. Furthermore, many of the sensor systems of the past for detecting physiological characteristics employ data and/or power lines that extend from the electrodes to the processor or power supply. These lines unavoidably get in the way of normal activity, even during monitoring of a sleeping subject body. Remote power packs worn by the user and portable circuitry have provided mobility to the subject body that is being monitored. However, the power packs still have lines to the circuitry and both the circuitry and power pack are rigid and bulky. Hence, the sensor systems of the past are deficient in eliminating power and data lines, and in reducing rigid and bulky elements.

### SUMMARY OF THE INVENTION

The instant invention overcomes the deficiencies of the past and fills a long standing need with a sensor system comprising a thin flexible ambulatory/self contained bio-sensor module. The sensor system includes the sensor module for collecting data during monitoring and a receiver module for receiving, processing, analyzing, and storing the data. The sensor module transmits the collected data by an integral RF transmitter or by a physically connectable integral port of the sensor module, which is removably connected to the receiver module for downloading after monitoring.

The sensor module can be made in appearance similar to an adhesive bandage and can be analogously adhered to the skin of a subject body in a convenient manner. The sensor module has no wires or cords extending to monitoring/receiving equipment. Therefore, there are no encumbering lines to entangle the limbs and torso of a subject body being monitored. As such, the subject body is free to walk and move about without interrupting the monitoring process, and without the sensor system or module interrupting the normal activities of the subject body. In this way, the subject person's bodily characteristics that are being monitored are closer to, if not the same as, what they would be during regular activities. Thus, the characteristics being monitored will more accurately reflect those characteristics that the subject body normally has, and will record the physiological responses of the subject body to his or her normal environment.

In this way the instant invention to be monitored outside the unnatural environment of a clinic or hospital or to be monitored more conveniently in such an environment. The bio-sensor system of the instant invention permits a subject person to not only be at home or work, but also to remain unencumbered by cords or battery packs. The sensor module is self-contained and has a low profile. Even with the added bulk of the thin flexible battery for the power supply, the sensor module is approximately as convenient as wearing an adhesive bandage.

The small size and wireless aspects of the instant sensor module avoid the noise that normally sullies the signal of past systems. The systems of the past have more noise because they have longer circuit paths between the monitoring circuitry and the physiological interface elements and longer circuit paths between the monitoring circuitry and the noise creating power supplies. On the other hand, the sensor module has an integral power supply such that the signal remains clean and free from noise. Thus, the sensor module and system overcome the deficiency of past systems including the inability of past systems to sense signals of very small amplitude. In other words, the instant sensor module and system are extremely sensitive and can accurately detect electromagnetic or other physiologically modulated signals from the body that heretofore were not possible to accurately detect due to the noise levels that are normally present in the conventional systems.

In summary, the instant sensor system includes a flexible sensor module and a receiver module. The flexible sensor module functions as a wireless or “untethered” data collection device that can be attached on the skin of a subject body and collect physiological data without need for physical connection to other electronics during the collection period. Alternatively, the flexible sensor module can be conformed to any of a variety of curved surfaces due its flexibility. The sensor module has a flexible thin sheet of silicon comprising circuitry, a flexible power source, and a flexible support substrate. The silicon, power source, and flexible substrate are integrated as layers of the sensor module. The layers can be placed together in the form of an adhesive bandage. A physiological interface element is connected to the sensor module. Typically, the physiological interface element comprises electrodes that protrude from the flexible substrate for contacting the skin of a subject body. However, other physiological interface elements such as optical sources and associated detectors can be implemented as well. The sensor system also includes one of an RF receiver with a wireless port for continuously or intermittently receiving data, or a physical port from which the sensor module is accessed for intermittently downloading data that is temporarily stored in a suitable memory contained in the sensor module.

It has been contemplated that the receiver module can comprise an intermediate transceiver separate from a subsequent destination of the data processing and analysis device. The intermediate transceiver could be worn on the subject body remote from the sensor module, for example. Signals from multiple sensor modules can be received in the intermediate transceiver and rearranged in time or otherwise processed to form a composite signal that is retransmitted to the data processing and analysis device as a single signal. The intermediate transceiver can make the data conform to the wireless Internet transmission standard or some other standard.

In one aspect of the invention, the sensor module further comprises a single integrated circuit on an active surface of the flexible thin sheet of silicon. Alternatively, the sensor module further includes a plurality of integrated circuits on an active surface of one or more thin sheets of silicon, and metallization on a surface of the flexible substrate facing the thin sheet of silicon. The metallization connects the circuits to each other. Each of the layers including the silicon is separately flexible along a respective entire width and entire length. Each of the layers remains flexible along the respective width and entire length when the sensor module is in its assembled configuration.

The sensor system includes at least one sensor module and at least one physically separate data storage and/or analysis device. Typically, the system will include a plurality of similar sensor modules to be simultaneously placed at a variety of selected locations on the skin of the subject body for simultaneously collecting data by each of the sensor modules.

In another aspect, the sensor system can be characterized as a bio data monitoring system having a sensor module in the form of a thin, flexible bio data collection device. The sensor module has a length, a width, and a thickness. The sensor module also has a plurality of layers of materials including a silicon layer. The layers are stacked in a thickness direction. Each of the layers is flexible and bendable out of a regular plane of the sensor module about both a lengthwise axis and a widthwise axis. The bio data monitoring system further has a data receiving and processing device that is physically separate from the sensor module as described above.

In another aspect, the layers of the sensor module include a nonconductive flexible substrate, a silicon layer in the form of a thin flexible sheet of silicon comprising an integrated circuit, and a bonding layer of anisotropic epoxy that bonds the silicon to the flexible substrate. In this aspect, the sensor module includes electrodes on a surface of the flexible substrate opposite the anisotropic epoxy layer. The sensor module also has metallization on the flexible substrate connecting the electrodes to the integrated circuitry.

An aspect of the invention in one of its simplest forms is an improved biomedical sensor module suitable for application to the skin of a subject body, in which the improvement over past devices comprises a flexible thin sheet of silicon comprising circuitry.

Another aspect, in accordance with the instant invention, includes a method of monitoring a physiological characteristic. One of the steps of this method is positioning a flexible, sensor module on the skin of a subject body to be monitored. After positioning the sensor module, data is collected through the skin for a predetermined period of time. Then the data is analyzed on a device that is physically separate from the sensor module. It should be noted that the steps of collecting and analyzing can further comprise monitoring by at least one of EKG, EMG, EEG, blood sugar, blood pulse, or blood pressure.

In accordance with this aspect, the step of positioning can comprise adhering the sensor module to the skin by adhesive pads on the sensor module. The method of monitoring can further include subsequent steps of removing the adhesive pads from the sensor module and heating the sensor module in an autoclave for sterilization after the data has been transferred to the receiver module.

Another aspect, in accordance with the instant invention, includes a method of making a sensor system comprising a flexible sensor module. The method of of making the system includes the method of making the flexible sensor module. The method of making the flexible sensor module comprises grinding an inactive side of a silicon layer on which an IC resides until the silicon becomes thin and flexible. The sensor module is further formed by mounting the silicon layer on a flexible substrate of polyimide by an anisotropic conductive epoxy intermediate layer. Another layer is added by covering the silicon layer and the flexible substrate with a thin flexible battery. The method of making the sensor system includes providing a data analysis device for processing the data. It should be noted that the instant invention advantageously entails thinning the silicon layer to a degree at which the fracture strength actually increases with decreasing thickness. In this way the instant invention overcomes the deficiencies of cracking and breaking of thin silicon that is expected as silicon becomes increasingly thin. Relatedly, the instant invention overcomes the need for stiff substrate material that is normally used to support thin silicon to prevent breaking of the silicon and destruction of any micro-circuitry thereon.

In one aspect of the invention, the sensor system is a bio data monitoring kit with at least one flexible sensor module as described above. In this kit and in all of the embodiments, the electrodes can be rigid or flexible. All of the other elements of the sensor module are flexible. In the case of a kit that includes a whole sensor system, the kit can include a separate data receiving and/or processing and analysis device. Furthermore, the system kit will include software for downloading onto the processor. A kit, moreover, can include one or more sensor module in the form of adhesive bandages.

### BRIEF DESCRIPTION OF THE DRAWINGS

FIG. 1 is a top plan view of an exemplary sensor module in accordance with the invention;

FIG. 2a is a schematic representation of the sensor system in accordance with the preferred embodiment of the invention;

FIG. 2b is a schematic representation of the sensor system in accordance with an alternative embodiment of the invention;

FIG. 2c is a perspective view of software for the system;

FIG. 3a is a perspective view of the sensor module;

FIG. 3b is an exploded perspective view of the sensor module;

FIG. 3c is a sectional view taken along lines 3c-3c of FIG. 3a;

FIG. 3d is a sectional view taken along lines 3d-3d of FIG. 3a;

FIG. 3e is a perspective view of the sensor module in a twisted configuration;

FIG. 3f is a cross sectional view taken along lines 3f-3f in FIG. 3b;

FIG. 4 is a graph depicting the relationship between fracture strength and thickness;

FIG. 5 is a schematic representation of internal stresses on an element experiencing bending forces;

FIG. 6 is an exemplary edge view showing the flexibility of thinned silicon;

FIG. 7 is a top plan view of the sensor module adhered at a specific location on the skin;

FIG. 8 is a perspective schematic representation of the placement of a sensor module in accordance with the invention in a probe; and

FIG. 9 is a block diagram of the circuitry that may be integrated onto the thinned silicon substrate.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS

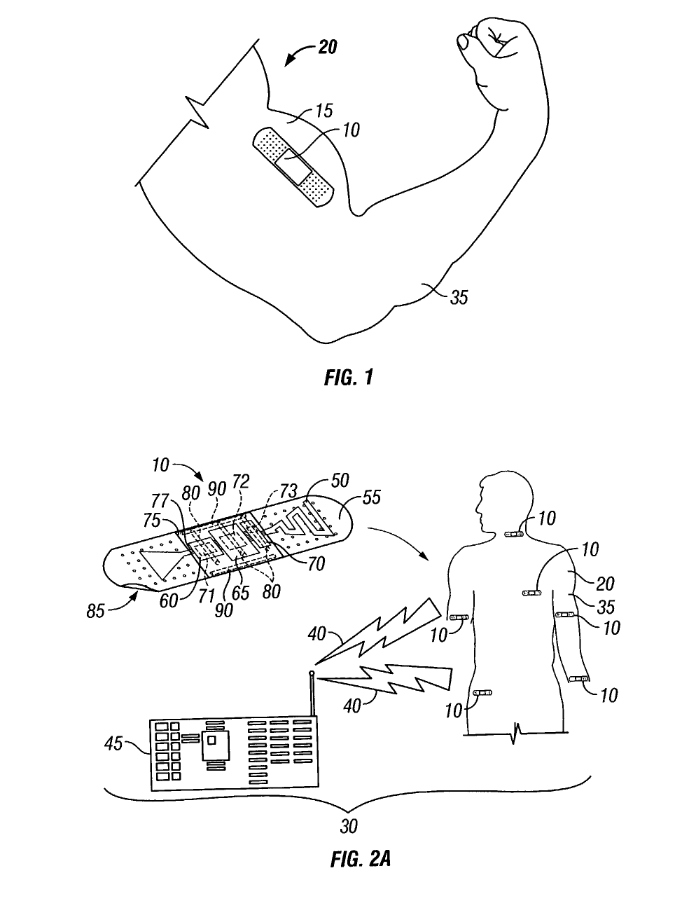

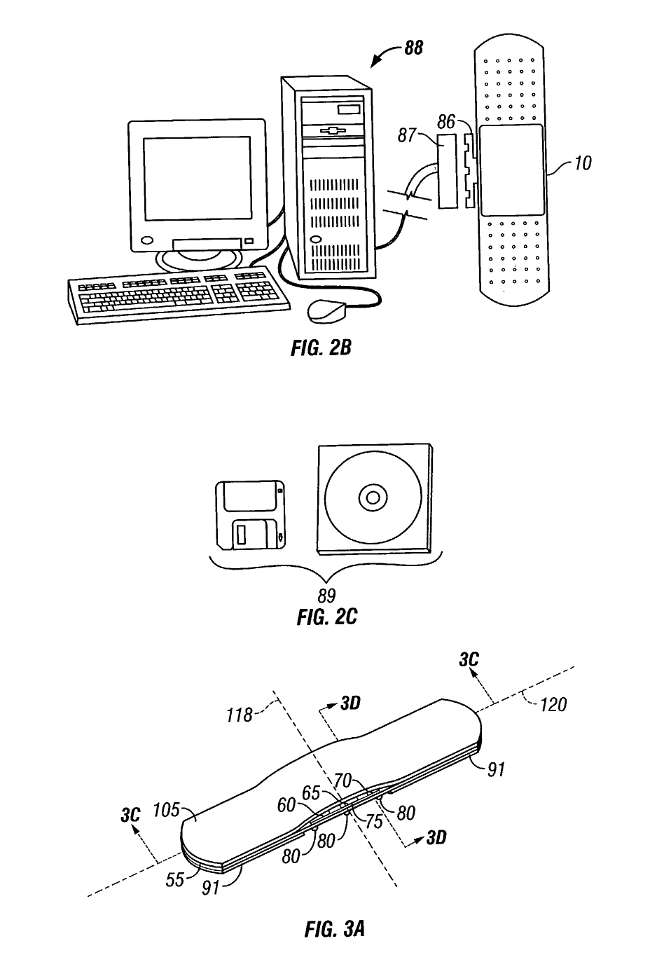

FIG. 1 shows an exemplary preferred embodiment of a sensor module 10 adhered to the skin 15 of a subject body 20 to be monitored. The sensor module 10 can be a single sensor module, (or one of a plurality of sensor modules), of the sensor system 30 shown in FIG. 2a in accordance with a preferred embodiment of the invention. As can be appreciated from FIG. 1, the sensor module 10 is completely unobtrusive, is self-contained, and is void of the monitor wires and other encumbrances that have conventionally entangled arms 35 and other parts of the subject body 20 during monitoring.

As shown in FIG. 2a, the system 30 can incorporate continuous transmission by RF signals 40 to a device 45 having a receiver, data storage, and/or means for analyzing the data. In this case, the sensor module 10 has at least one antenna 50 metalized onto a flexible substrate 55 for transmitting and/or receiving RF signals. As shown in FIG. 2a, thin flexible silicon substrates 60, 65, 70 having respective integrated circuits 71, 72, 73 are bonded to the flexible substrate 55 by an anisotropic epoxy layer 75 or the like. As can be appreciated, the metallization 77 is applied to the flexible substrate 55 and extends between the anisotropic layer 75 and the flexible substrate 55. The metallization 77 interconnects the ICs 71, 72, 73 to each other and to the antenna 50. The metallization 77 further extends through the flexible substrate 55 and connects the ICs 71, 72, 73 to the electrodes 80 disposed on an underside 85 of the flexible substrate 55 for contact with the skin 15 of the subject body 20. The preferred metalization 77 is formed during one or more process steps or from discrete conductors.

While the preferred embodiment comprises RF continuous transmission of signals 40 to a remote device 45 during monitoring, an alternative embodiment shown in FIG. 2b incorporates memory in the ICs 71, 72, 73 for storing data during monitoring. This alternative embodiment includes a port 86 on the sensor module 10 and a mating connection 87 connected to a PC 88 for physically connecting the sensor module 10 to the PC 88 or other processor for downloading, analyzing, and archiving collected data. The memory device can be a non-volatile memory in case power is lost to the sensor module.

For the embodiments of both FIG. 2a and 2b, the system includes software 89 for controlling input, storage, and analysis of the collected data on the processor 88. The software may be provided on a CD, floppy, or any storage media as generally indicated in FIG. 2c. It is contemplated that the invention can be provided as a kit having one or more of the components of the system 30, and may include software 89. Of course, the software 89 will be included in kits that comprise the complete sensor system 30.

While the silicon substrates 60, 65, 70 are shown separately with separate respective ICs 71, 72, 73, it is to be expressly understood that the ICs 71, 72, 73, can be integrated as one IC on a single silicon substrate. Doing this would provide cost advantages. Furthermore, a single circuit could incorporate both of the embodiments so that a user could selectively implement monitoring by continuous RF transmission, or by storage and subsequent retrieval by physical connection of the sensor module to the PC 88 or the like for downloading data captured during a period of monitoring.

Furthermore, it is to be understood that while in the preferred embodiment, the electrodes 80 are oriented to extend longitudinally in a width-wise direction of the sensor module 10, other types, configurations, and orientations of electrodes are considered to be within the spirit and scope of the invention. Furthermore, orienting a pair of electrodes 90 to extend longitudinally relative to the sensor module 10 and positioning them along lateral edges as shown in FIG. 2 has been contemplated. In addition, other types of physiological data detectors can be used.

Of course, more than one sensor module 10 can be selectively located at a variety of positions on the body 20. In the preferred embodiment, the sensor system 30 detects bioelectric signals created by muscle movement and transmits the signals 40 in support of EMG monitoring. In accordance with the invention, the circuits 71, 72, 73 are configured to detect very small electromagnetic signals. Furthermore, by changing the input filter characteristics and/or the physiological interface element(s), a similar sensor module can be used to detect and digitize other physiological characteristics such as an EKG (from the heart), an EEG (from the brain), pulse rate , blood pressure, and blood sugar levels. Other techniques such as new non-linear techniques for analyzing the frequency spectrum of the EMG signal can be incorporated to monitor other physiological characteristics such as during non-static contractions of muscles. These techniques can be used to detect the presence of foreign chemicals in the subject body 20. As can be appreciated, different physiological characteristics can be simultaneously monitored at respective positions on the body 20.

Because of the thin profile, flexibility, and stand-alone/ambulatory nature of the sensor module 10, the subject body 20 can be monitored during normal activity without interruption or discomfort. Furthermore, since the sensor module 10 is unobtrusive and ambulatory, monitoring can be carried out without signal abnormalities due to subject reaction to irritation from the device. Still further, the sensor module 10 avoids the signals being adversely affected by subject reaction to the unnatural environment in which conventional monitoring is carried out. Due to the flexibility, the electrode positioning, and the secure adhesive bandage configuration of the sensor module, signal distortion during muscle contraction is also avoided. This is because the electrodes are held secured against separation from the skin 15 by the adhesive pads 91. Signal distortion is also mitigated because the flexibility of the sensor module permits continuous contact by the electrodes, even during changes in the contour of the skin 15 due, for example, to contraction of the underlying muscles.

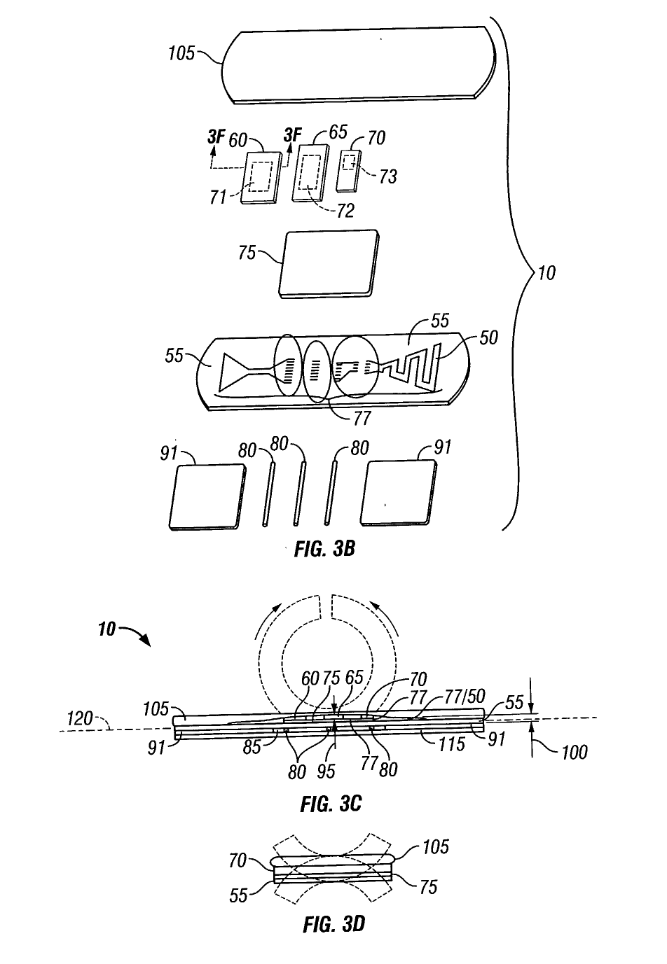

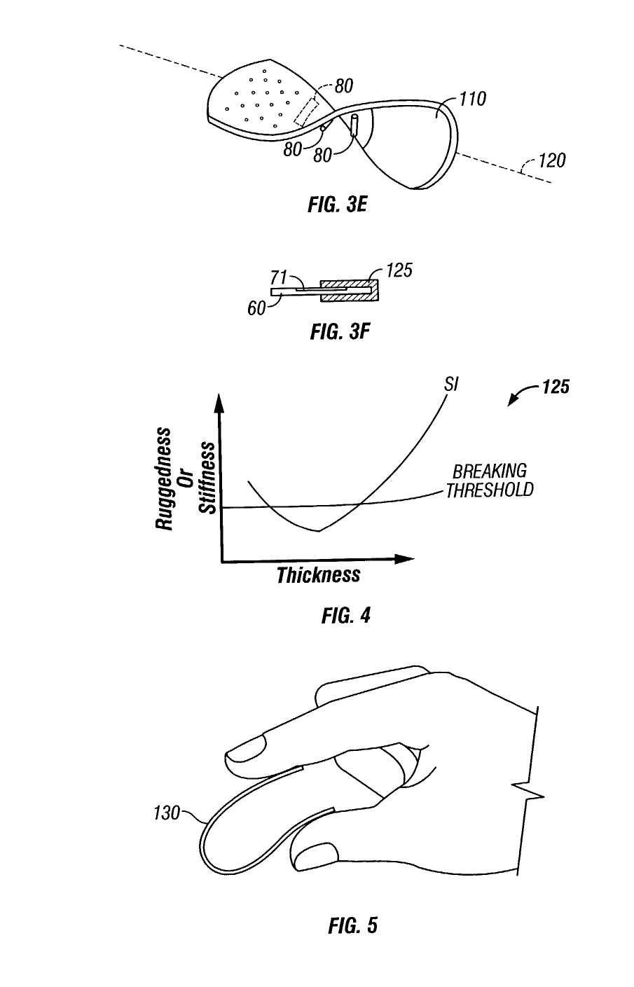

The perspective view of the sensor module 10 shown in FIG. 3a illustrates the thin structure of the various layers, and of the overall sensor module 10. The flexible substrate 55 is preferably in the form of a polyimide, and is electrically non-conductive and flexible. However other flexible non-conducting substrates can be substituted. The flexible substrate 55 supports the other layers of the sensor module 10. This is perhaps best shown in FIGS. 3b and 3c. For example, the next superjacent layer is the anisotropic epoxy 75 shown in the sectional view of FIG. 3c. Disposed between portions of the flexible substrate 55 and the anisotropic epoxy 75 is metallization 77, which is best shown in the exploded perspective view of FIG. 3b. The next superjacent layer to the anisotropic epoxy 75 is the thin flexible silicon 60, 65, 70. The silicon 60, 65, 70 is bonded to the flexible substrate 55 by the anisotropic epoxy layer 75. The anisotropic epoxy layer 75 has properties preventing electrical conduction therethrough in one direction while permitting electrical conduction therethrough in the other direction.

As shown in FIG. 3c, the thickness 95 of the thin silicon substrates 60, 65, 70 is in the range from 10 to 50 microns. The preferred thickness is approximately 25 microns since it is roughly in the middle of the operable range. The sum 100 of the thicknesses of the flexible substrate 55, the metallization 77, the anisotropic epoxy 75, and the silicon substrates 60, 65, 70, is in the range in from 75 to 100 microns. The metallization 77 is typically integral with and forms part of the layer of thee flexible substrate 55.

A flexible, thin battery 105 overlays the silicon substrates 60, 65, 70 and their respective ICs 71, 72, 73. As shown in FIGS. 3a-3c, the battery 105 preferably also covers the flexible substrate 55 and the metallization 77. The battery is connected to the ICs by the metallization 77. The electrodes 80 are located on a lower surface 85 of the flexible substrate 55 and are connected through the flexible substrate 55 to the metallization 77. The metallization 77 in turn connects the electrodes 80 to the ICs 71, 72, 73. The electrodes 80 may comprise any of a variety of electrically conductive materials. However, in the preferred embodiment, the electrodes 80 are formed of silver wire.

Adhesive pads 91 are also disposed on the underside 85 of the flexible substrate. The adhesive pads 91 are located analogously to adhesive portions of a conventional adhesive bandage. The adhesive pads 91 and electrodes 80 can be covered by a peel away or other protective cover 115 in a conventional manner. Furthermore, the adhesive pads 91 can be formed of double-sided adhesive for application before and removal after each use so that the sensor module can be used repeatedly. The sensor module can be sterilized in an autoclave, for example, between uses. Alternatively, the sensor modules are made disposable and are discarded after monitoring a particular subject body 20 for purposes of good hygiene.

All of the materials and layers described above in relation to FIGS. 3a-3c are flexible except for the electrodes 80. The elements remain flexible when assembled together as indicated by the dashed lines of FIGS. 3c and 3d, which show bent configurations of the sensor module 10. The dashed lines in FIG. 3c depict bending about a lateral axis 118 shown in FIG. 3a. The lateral axis extends orthogonally to a longitudinal axis 120 also shown in FIG. 3a. The dashed lines of FIG. 3d depict bending about the longitudinal axis 120. Bending about these axes 118, 120 is facilitated by all of the materials except for the electrodes. However, the extension of the electrodes 80 in a length-wise direction is minimal as shown in FIG. 3c. Hence, bending about the lateral axis 118 is substantially not inhibited by the electrodes 80. Bending about the longitudinal axis 120 is only inhibited slightly in regions where the electrodes 80 are connected to the sensor module 10. Furthermore, the electrodes 80 are spaced far enough apart to permit flexure about both a longitudinal and a transverse axis, and twisting of the sensor module about a longitudinal axis 120, for example, as shown in FIG. 3e.

Preferably, the active side of the silicon sheets 60, 65, 70 faces the anisotropic layer 75. However, it is possible to provide the active side facing away from the anisotropic layer 75. In this case it is necessary to provide reroute metallization 125 from the circuits 71, 72, 73 around an edge of the silicon layer and to selected locations on an inactive side of the silicon 60, 65, 70 as shown, for example, in FIG. 3f. The reroute metallization 125 provides electrical connections from the circuits 71, 72, 73 to selected positions on the anisotropic layer 75, which in turn provide electrical connections with metallization 77 on the flexible substrate 55.

By way of example and not by way of limitation, the length of the sensor module is in the range from 30 to 60 mm, and the width is in the range from 10 to 20 mm. The regularly small width dimension of the sensor module 10 further renders the localized inhibition of bending of little consequence. Alternatively, the electrodes can be made of a flexible material or can be modified to be shorter than is depicted in the Figures.

Most of the materials for the sensor module can be selected from a variety of available flexible materials. However, the material on which the ICs are formed is more limited. While organic polymer semi-conducting substrates can be used to provide flexibility, they are not the preferred material. This is because they do not have consistent and uniform electronic properties throughout the substrate. The superior electronic properties of the crystalline-structured, traditional silicon semiconductors are preferred. At conventional thicknesses, however, silicon is rigid and not flexible. Hence, utilization of a silicon substrate in its conventional form would defeat the purpose of providing a flexible sensor module in accordance with the instant invention.

However, in accordance with the graph shown in FIG. 4, the fracture strength of silicon actually increases with decreasing thickness for a certain range of low thicknesses. The instant invention takes advantage of this physical characteristic by thinning silicon to a range from 10 to 50 microns while maintaining the integrity of the integrated circuits 71, 72, 73 on an active surface of the silicon 60, 65, 70. As such, flexibility of a sheet of silicon 130 is achieved as shown in FIG. 5. The integrity of any integrated circuits on the silicon sheet 130 is maintained unless the silicon sheet 130 is actually folded.

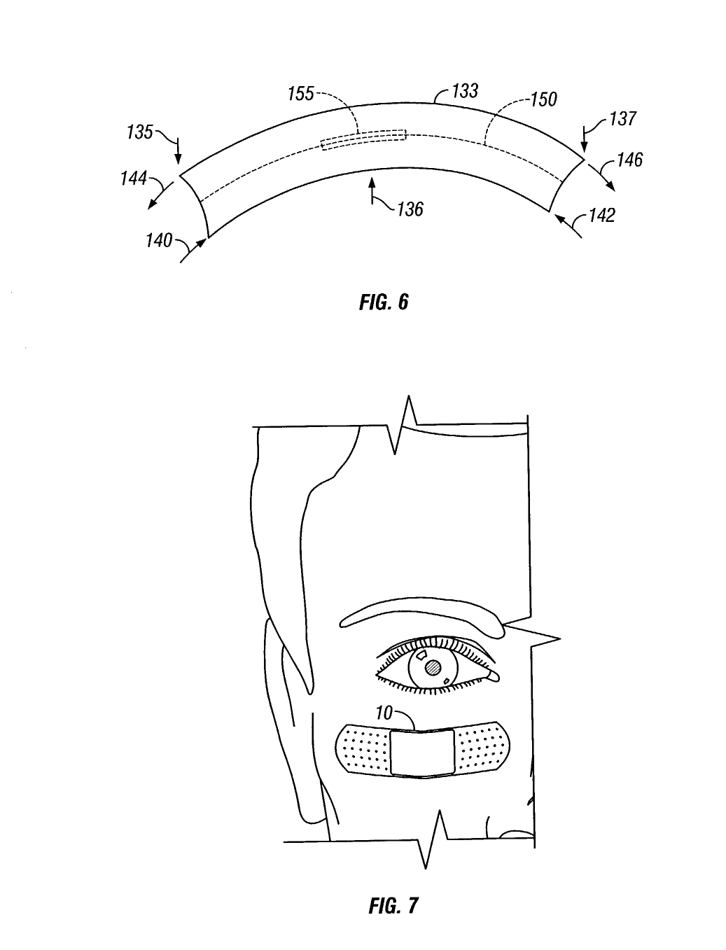

The instant invention also takes advantage of a mechanical property of many solids and laminants. This property is shown in FIG. 6. Simply stated, it is that when a solid or laminant 133 that is resistant to internal shearing is bent, internal shearing reaction forces are set up within the solid. For example, as shown in the FIG. 6, in response to external bending forces 135, 136, 137, internal shearing forces 140, 142, 144, 146 are set up inside the solid 133. As shown, there is a central plane, (or in two dimensions, a central line), called the zero stress plane 150 at which the internal shear forces are substantially zero. Hence, it can be seen that locating an element 155 at the central zero stress plane 150 has protective advantages. As shown in FIG. 3a, the silicon substrates 60, 65, 70 are located generally in a central plane. The overlying battery 105 takes up some of the internal stress as do the underlying flexible substrate 55 and the epoxy 75. Additional layers and specific materials can be added as needed to provide a zero stress plane substantially through the silicon substrates 60, 65, 70. Specifically, it has been found that an addition of a polyimide coating on an upper surface of the silicon substrates 60, 65, 70 can sometimes help to center the silicon substrates 60, 65, 70 on the zero stress plane 150.

FIG. 7 shows an exemplary application of the sensor module 10 at a specific location on a subject body 20. In this location, the muscle movement that opens and closes the eyelid is monitored.

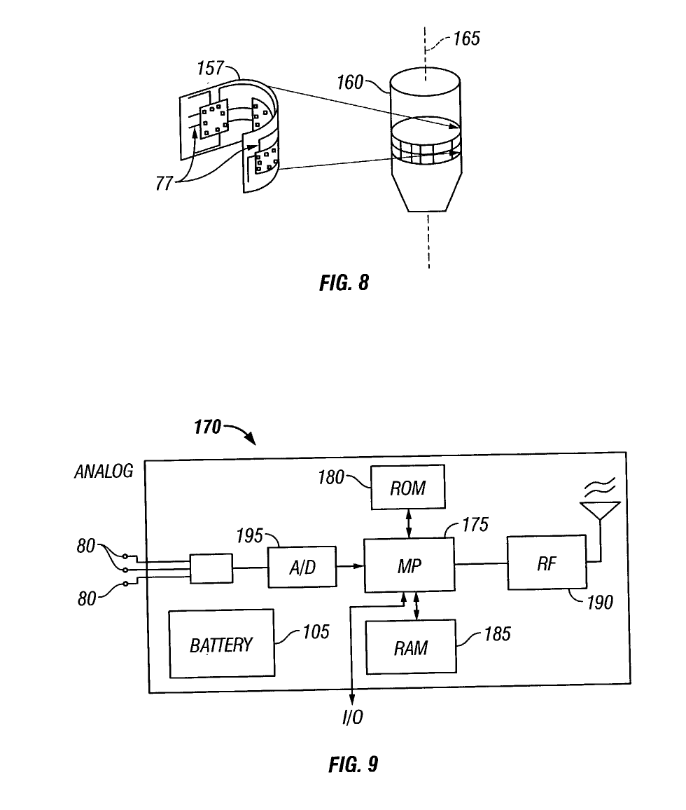

FIG. 8. depicts a sensor module 157 that is uniquely used to form part of a probe 160. Since the sensor module 10 is flexible, it can be conformed to a curved surface like the cylinder of probe 160. As shown, the sensor module 157 is oriented so that the plane or curve of the device is parallel to the longitudinal axis 165 of the probe 160. In this particular case, the probe has a cylindrical, protective shell into which the sensor module preferably can be inserted in a rolled configuration. This embodiment can be specifically applied with the sensor module 157 in the form of a pill to be swallowed, a probe for insertion, or a bullet that can penetrate a subject body, for example.

FIG. 9 is a block diagram showing circuitry 170 that could be incorporated into the thin silicon substrates 60, 65, 70. The precise circuitry configuration is not critical to an understanding of the invention. However, it is to be generally understood that the circuitry will include a microprocessor 175, a ROM 180 for storing a program to be implemented, a RAM 185 for storing of data, and a transmitter or transceiver 190 for transmitting data by RF signals to a remote receiver. An optional I/O interface could be used instead of or in addition to the transmitter 190. The circuitry also includes the electrodes 80 for front end analog data collection. The electrodes 80 are connected to an analog/digital converter 195 to convert the analog signals to digital signals to be processed in the microprocessor 175.

Many alterations and modifications may be made by those having ordinary skill in the art without departing from the spirit and scope of the invention. Therefore, it must be understood that the illustrated embodiments have been set forth only for the purposes of example and that it should not be taken as limiting the invention as defined by the following claims. The claims are thus to be understood to include what is specifically illustrated and described above, what is conceptionally equivalent, what can be obviously substituted and also what incorporates the essential idea of the invention.

What is claimed is:

1. A sensor module that functions as a wireless data collection device, comprising:
a flexible thin sheet of silicon comprising circuitry for collecting physiological data, one of a wireless port for continuously transmitting data or a port for intermittently uploading stored data, a flexible power source, and a flexible substrate integrated as layers of the sensor module in the form of an adhesive bandage; and
a plurality of the electrodes protruding from the flexible substrate for contacting the skin of a person.

2. The sensor module of claim 1, further comprising a bonding layer comprising an anisotropic conductive epoxy bonding the flexible thin sheet of silicon to the flexible substrate.

3. The sensor module of claim 2, wherein each of the layers is flexible along a width and along an entire length.

4. The sensor module of claim 2, wherein a thickness of the silicon, the epoxy, and the flexible substrate layers is in the range from 75 to 100 microns.

5. The sensor module of claim 1, wherein a circuit path and the power source comprise means for reducing noise.

6. The sensor module of claim 1, wherein a thickness of the thin sheet of silicon is in the range from 10 to 50 microns.

7. The sensor module of claim 6, wherein the thickness of the thin sheet of silicon is approximately 25 microns.

8. The sensor module of claim 1, wherein the sensor module is one of a plurality of similar sensor modules to be simultaneously placed at a variety of selected locations on the skin of the person.

9. The sensor module of claim 1, further comprising a single integrated circuit on an active surface of the flexible thin sheet of silicon.

10. The sensor module of claim 1, further comprising:
a plurality of flexible thin sheets of silicon that each have integrated circuits on an active surface thereof; and
metallization connecting the integrated circuits to each other.

11. The sensor module of claim 1, further comprising metallization on a surface of the flexible substrate facing the flexible thin sheet of silicon, the metallization forming an antenna.

12. The sensor module of claim 1, wherein:
the flexible substrate is a polyimide and is located on a first side of the flexible thin sheet of silicon; and
the power source is a thin battery and is located on a second side of the flexible thin sheet of silicon opposite to the flexible substrate and thereby helps to center the flexible thin sheet of silicon on a zero stress plane of the sensor module.

13. The sensor module of claim 1, further comprising a plurality of adhesive pads on the flexible substrate for attaching the sensor module to the skin.

14. The sensor module of claim 13, wherein the adhesive pads are double sided and removably attached to the flexible substrate so that the adhesive pads can be removed from the sensor module after use, and the sensor module can be sterilized in an autoclave for subsequent attachment of new adhesive pads and repeated usage.

15. A sensor system, comprising:
a sensor module with:
a flexible thin sheet of silicon comprising circuitry
and a flexible substrate integrated as layers of the sensor module; and
a receiver module that is physically separate from the sensor module during monitoring by the sensor module, the receiver module comprising one of:
an RF receiver with a wireless port for continuously receiving data, or
a port for physically connecting to and downloading stored data from the sensor module.

16. A thin, flexible sensor module, comprising:
a length, a width, and a thickness; and
a plurality of layers of materials including a silicon layer, the layers stacked in a thickness direction, wherein each of the layers is flexible and bendable out of a regular plane of the sensor module about both of a lengthwise axis and a widthwise axis.

17. The sensor module of claim 16, wherein the layers include:
a nonconductive flexible substrate;
the silicon layer comprising a thin flexible sheet of silicon comprising an integrated circuit; and
a bonding layer of anisotropic epoxy bonding the silicon to the flexible substrate.

18. The sensor module of claim 17, wherein an overall thickness of the layers is less than or equal to 100 microns.

19. The sensor module of claim 17, wherein the thin flexible sheet of silicon has a thickness in the range from 10 to 50 microns.

20. The sensor module of claim 17, further comprising:
electrodes on a surface of the flexible substrate opposite the anisotropic epoxy layer; and
metallization on the flexible substrate connecting the electrodes to the integrated circuit.

21. A sensor system, comprising:
a thin, flexible sensor module having a plurality of layers of materials including a silicon layer, the layers stacked in a thickness direction, wherein each of the layers is flexible and bendable in a thickness direction out of a regular plane of the sensor module;
a receiver module having a data receiving and processing device that is physically separate from the sensor module during monitoring by the sensor module.

22. The sensor system of claim 21 further comprising an intermediate transceiver that is separate from the receiver module.

23. The sensor system of claim 22 further comprising a plurality of sensor modules and wherein the intermediate transceiver receives signals from the plurality of sensor modules, rearranges such signals in time to form a composite signal, and then transmits the composite signal to the receiver module.

24. The sensor system of claim 22 further comprising a plurality of sensor modules and wherein the intermediate transceiver receives signals from the multiple sensor modules, process such signals to form a composite signal that conforms to a communication standard, and then transmits the composite signal to the receiver module.

25. An improved biomedical sensor module suitable for application to the skin of a subject body, the improvement comprising:
a flexible thin sheet of silicon comprising circuitry.

26. A method of monitoring a physiological characteristic, comprising:
positioning a flexible, sensor module on the skin of a subject body to be monitored;
collecting data through the skin for a predetermined period of time; and
analyzing the data on a device that is physically separate from the sensor module.

27. The method of monitoring of claim 26, wherein the step of positioning comprises adhering the sensor module to the skin by adhesive pads on the sensor module.

28. The method of monitoring of claim 27, further comprising the step of discarding the sensor module after use on a single said subject body.

29. The method of monitoring of claim 27, further comprising the subsequent steps of:
removing the adhesive pads from the sensor module; and
heating the sensor module in an autoclave for sterilization.

30. The method of monitoring of claim 26, wherein:
the step of positioning further comprises locating the sensor module in any of a variety of positions on the skin; and
the step of collecting further comprises comfortably leaving the sensor module on the skin during normal activities of the subject body.

31. The method of monitoring of claim 26, wherein the steps of collecting and analyzing further comprise monitoring by EMG.

32. The method of monitoring of claim 26, wherein the steps of collecting and analyzing further comprise monitoring by at least one of EKG, EMG, EEG, blood sugar, blood pulse, or blood pressure.

33. The method of monitoring of claim 32, wherein:
the sensor module is one of a plurality of sensor modules, and further comprising:
positioning the plurality of sensor modules on selected positions on the skin; and
simultaneously collecting data by each of the sensor modules.

34. A method of making a flexible sensor module, comprising:
grinding an inactive side of a silicon layer on which an IC resides until the silicon becomes thin and flexible;
mounting the silicon layer on a flexible substrate of polyimide by an anisotropic conductive epoxy intermediate layer; and
covering the silicon layer and the flexible substrate with a thin flexible battery.

35. The method of making of claim 34, wherein the step of grinding further comprises thinning the silicon layer to a thickness in the range from 10 to 50 microns.

36. The method of making of claim 34, further comprising:
metalizing the flexible substrate in order to:
provide an antenna; and
connect the IC to electrodes.

37. A method of making a sensor system, comprising:
forming a sensor module by:
grinding an inactive side of a silicon layer on which an IC resides until the silicon becomes thin and flexible; and
mounting the silicon layer on a flexible substrate; and
providing a receiver module in the form of a data processing device for analyzing the data.

38. A bio data monitoring system kit, comprising:
at least one wireless, flexible sensor module having a thinned silicone layer with an IC and a plurality of electrodes thereon; wherein:
the sensor module has an adhesive bandage configuration; the electrodes are rigid or flexible; and
all of the other elements of the sensor module are flexible.

39. The kit of claim 38, further comprising software for controlling collection, analysis, and storage of the data received from the at least one sensor module.

40. The kit of claim 38, further comprising a receiver for receiving data collected by the sensor module.
